(19) United States
(12) Patent Application Publication
(10) Pub. No.: US 2013/0066415 A1
(43) Pub. Date: Mar. 14, 2013

(54) STENT

(71) Applicant: Gordon Donald Hocking, Chiba (JP)

(72) Inventor: Gordon Donald Hocking, Chiba (JP)

(21) Appl. No.: 13/698,846

(22) PCT Filed: May 25, 2011
(86) PCT No.: PCT/JP2011/002903

§ 371 Date: Nov. 19, 2012

(30) Foreign Application Priority Data
May 25, 2010 (JP) 2010-119346

(51) Int. Cl. A61F 2/90

(52) U.S. Cl. 623/1.15

(57) ABSTRACT

To provide a stent for enabling injuries to the wall of a lumen to be reduced, a stent 10 in the shape of a mesh is placed and used inside a lumen such as a blood vessel of the human body, where the mesh is formed of a single or plurality of pieces of wire material 11 having elasticity, and the wire material 11 bends in the shape of a curve without being folded in end portions 10A, 10B of the stent 10, and is formed in the shape of a cylinder.

### TECHNICAL FIELD

The present invention relates to a stent that is placed inside a lumen of the human body to prevent the occurrence of occlusion inside the lumen and breakage of the lumen.

### BACKGROUND

Among cerebrovascular diseases, cerebral strokes that acutely occur abruptly are developed by occlusion of the cerebral blood vessel or bleeding from the cerebral blood vessel as a leading cause.

Among the strokes, the cerebral stroke developed by occlusion of the cerebral blood vessel is a disease that occurs when an embolus (as well as a thrombus, fat embolus, tumor embolus, etc.) enters the cerebral artery, develops stenosis of the artery, interrupts the blood flow and develops cerebral ischemia, and is called cerebral infarction. When the blood flow to the brain is interrupted by an embolus, brain cells are cut off from oxygen and nutrition, and become necrotic in a short time. Therefore, in the initial stage of occurrence of the cerebral infarction, it is important to secure the normal blood flow promptly. Unless the normal blood flow is secured early, the brain tissue loses the function of the site, and the risk becomes high that the life of a patient is threatened.

Meanwhile, cerebral strokes developed by bleeding from the cerebral blood vessel are classified into a cerebral hemorrhage and a subarachnoid hemorrhage according to the location of bleeding. Among the strokes, the subarachnoid hemorrhage is caused by rupture of a cerebral aneurysm developed on the wall of the cerebral artery in the majority of cases. The aneurysm lacks the tunica media in the wall of the blood vessel, and tends to cause a rupture, the subarachnoid hemorrhage has a characteristic of tending to recur once it occurs, and therefore, when an unruptured aneurysm is found, the cautious decision is required on whether or not to perform the treatment for preventing occurrence of rupture.

Previously, when it is necessary to perform therapy for occlusion of the cerebral blood vessel or the treatment for an aneurysm developed in the cerebral blood vessel as described above, the method has been adopted of directing operating the cerebral blood vessel by a craniotomy or cervical incision. Meanwhile, in recent years, with the progression of endovascular treatment techniques, non-invasive techniques with fewer loads on the body of a patient have attracted attention. Among the techniques, stent placement is one of well-known techniques.

The stent placement is techniques of placing a fine tube called the stent inside a lumen such as a blood vessel, thereby widening the stenosed site and preventing a rupture of the aneurysm. Generally, in the stent placement, a catheter is inserted from a blood vessel (femoral artery) of the groin or the like under local anesthesia, and through the catheter, the stent is carried to a lesion area where the aneurysm or stenosis occurs. As a method of placing, there are two methods of balloon-expanding type and self-expending type. In the case of balloon-expanding type, a balloon with the stent installed outside is expanded in the lesion area to widen the lesion area and the stent, then the balloon is only removed, and the stent is placed. Alternately, only the balloon is expanded in the stenosed site aside from the stent to widen, and the stent is placed in the stenosed site. Meanwhile, in the self-expanding type, when the stent is guided to the lesion area and unloaded, the stent expands automatically and is fitted with the wall of the blood vessel. By thus placing the stent inside the blood vessel, the stenosed site is expanded or the blood stream to the aneurysm is restricted.

FIG. 10 shows an example of the stent used in conventional stent placement. As shown in FIG. 10, generally, the conventional stent 100 is formed by making woven thin metal wires 101 having elasticity in the shape of a tube, and cutting in an appropriate size. Further, when necessary, a resin coating or the like is applied.

Further, as shown in FIG. 11, a stent 200 with wires 201 in end portions 200A, 200B welded and joined has conventionally been used.

### SUMMARY OF INVENTION

### Technical Problem

However, in the above-mentioned conventional stent 100, since the wires 101 made of metal are cut, wires 101 in the end portions 100A, 100B project in the shape of burrs. Further, in the conventional stent 200, the welded portions (end portions 200A, 200B) of the wires 201 protrude sharply. Therefore, in carrying or placing or after placing the stents 100, 200, the end portions 100A, 100B, 200A, 200B may injure the blood vessel. Therefore, there is the risk that an ulcer, perforate and/or bleeding may develop. Further, the injured blood vessel tries to form intimae as a healing reaction. When the formation of intimae is excessive, the blood vessel and inside of the stent are narrowed, the blood stream is interrupted, and there is a possibility that the serious condition is caused again. In order to cope with such excessive formation of intimae, it is necessary to replace the stent regularly.

In view of the above-mentioned circumstances, it is an object of the invention to provide a stent for enabling injuries to the wall of a lumen to be reduced.

### Solution to Problem

The invention provides a stent which is a stent in the shape of a mesh that is placed and used inside a lumen such as a blood vessel of the human body, and which is characterized in that the mesh is formed of a single or plurality of pieces of wire material having elasticity, and that the wire material bends in the shape of a curve without being folded in end portions of the stent, and is formed in the shape of a cylinder.

Herein, it is suitable that the end of the wire material is formed to be positioned in a side portion except the end portions of the shape of a cylinder.

Further, the stent of the invention may be formed in the shape of a cylinder in which diameters are almost the same over from one end portion to the other end portion. Alternately, the stent may be formed in an opposite-end broad shape in which diameters in opposite end portions are larger than a diameter of the side portion close to the center. Furthermore, the stent may be formed in a tapered shape in which the diameter in one end portion is larger than the diameter in the other end portion.

In addition, it is suitable that the wire material is a wire made of gold, platinum, or tungsten, or a wire comprised of nickel titanium coated with gold or platinum.

Further, it is suitable that the outside diameter of the shape of a cylinder is formed into 5 mm, and that the stent is placed and used inside the cerebral blood vessel.

### Advantageous Effects of Invention

According to the stent of invention, it is possible to prevent the wall of a lumen from being injured and to reduce the risk of stenosis of the lumen.

### BRIEF DESCRIPTION OF DRAWINGS

FIG. 1 is an entire schematic view showing the outline of a stent according to an Embodiment of the invention.

FIG. 2 is a schematic view showing an example of a state of a lesion area to use the stent as shown in FIG. 1.

FIG. 3 contains schematic views (part 1) showing an example of the method for using the stent as shown in FIG. 1.

FIG. 4 contains schematic views (part 2) showing an example of the method for using the stent as shown in FIG. 1.

FIG. 5 contains schematic views (part 3) showing an example of the method for using the stent as shown in FIG. 1.

FIG. 6 contains schematic views (part 4) showing an example of the method for using the stent as shown in FIG. 1.

FIG. 7 is a cross-sectional schematic view showing an example of a stent introducer.

FIG. 8 is an entire schematic view showing the outline of a stent (opposite-end broad shape) according to another Embodiment of the invention.

FIG. 9 is an entire schematic view showing the outline of a stent (tapered shape) according to still another Embodiment of the invention.

FIG. 10 is an entire schematic view showing an example of a stent used in conventional stent placement.

FIG. 11 is an entire schematic view showing another example of the stent used in conventional stent placement.

### DESCRIPTION OF EMBODIMENTS

Embodiments of the invention will specifically be described below with reference to drawings.

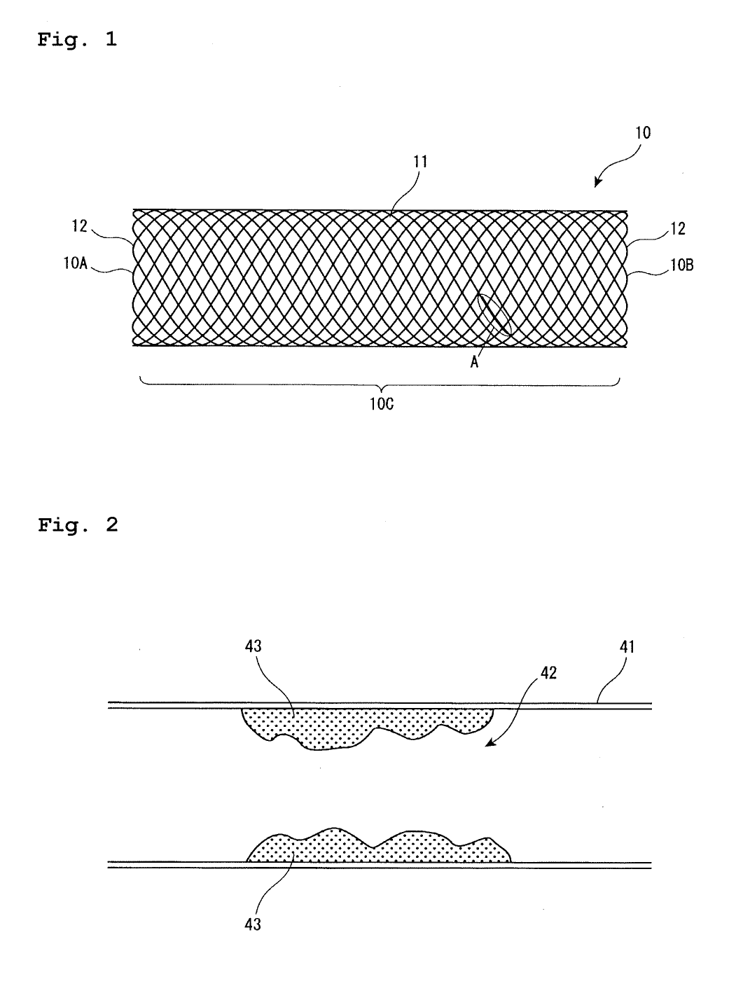

FIG. 1 is an entire schematic view showing the outline of a stent according to an Embodiment of the invention. As shown in FIG. 1, the stent 10 is configured by weaving a wire (wire material) 11, which is extremely thin and rich in flexibility and has elasticity, in the shape of a cylinder. Herein, the stent 10 maybe woven from a plurality of wires or woven from only a single wire. Then, the beginning and the end of the wire 11 are woven to be positioned in the side portion 10C located between the opposite end portions 10A, 10B, instead of being positioned in the end portions 10A, 10B of the stent 10 (see the A portion of FIG. 1). Further, in the opposite end portions 10A, 10B of the stent 10, the wire 11 forms curved portions 12 bent in the shape of a curve with roundness so as not to be folded and protrude.

Herein, as the wire 11 forming the stent 10, it is suitable to use a wire made of gold, platinum or tungsten, or a wire obtained by applying a gold or platinum coating to nickel titanium.

With respect to the method for using the above-mentioned stent 10, an example will be described below. In addition, herein, the description is given on the case of using the stent 10 in a stenosed site 42 of a cerebral blood vessel 41 by an embolus 43 as shown in FIG. 2 as an example. Further, in the following description, the case that the stent 10 is an automatic-expanding type will be described.

(1) Expansion of the Stenosed Site by a Balloon

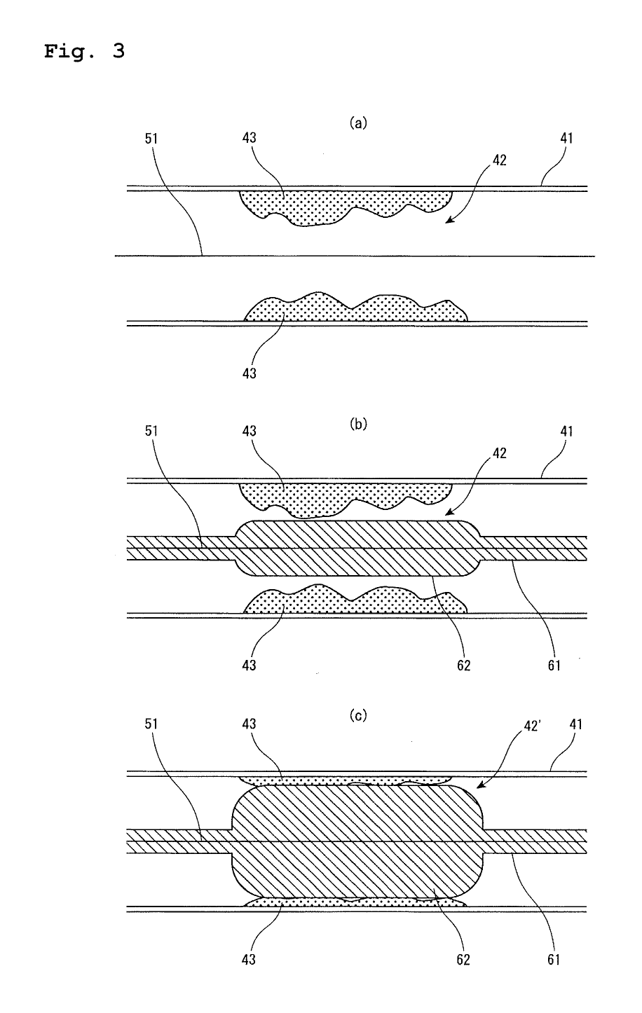

First, as shown in FIG. 3(a), a guide wire 51 is sent into the blood vessel 41 up to a position exceeding the stenosed site 42.

Then, as shown in FIG. 3(b), a balloon catheter 61 is passed onto the guide wire 51, is sent into the blood vessel 41, and shifted to a position such that a balloon portion 62 reaches the stenosed site 42.

Next, as shown in FIG. 3(c), air is blew to the balloon catheter 61 to expand the balloon portion 62, and the stenosed site 42 is enlarged. Subsequently, the balloon catheter 61 is removed.

(2) Insertion of a Carrier Catheter and Stent Introducer

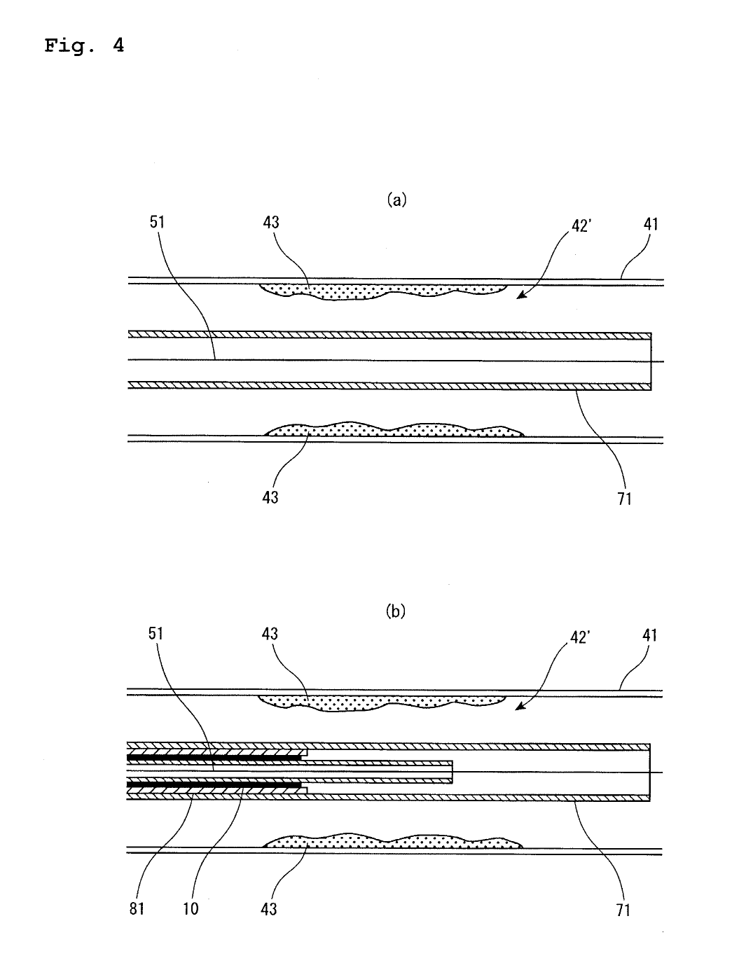

Next, as shown in FIG. 4(a), a carrier catheter 71 is passed onto the guide wire 51, and is sent to a position exceeding the enlarged stenosed site 42′.

Then, as shown in FIG. 4(b), a stent introducer 81 with the stent 10 beforehand stored therein is pierced with the guide wire 51 to move forward, and inserted into the carrier catheter 71.

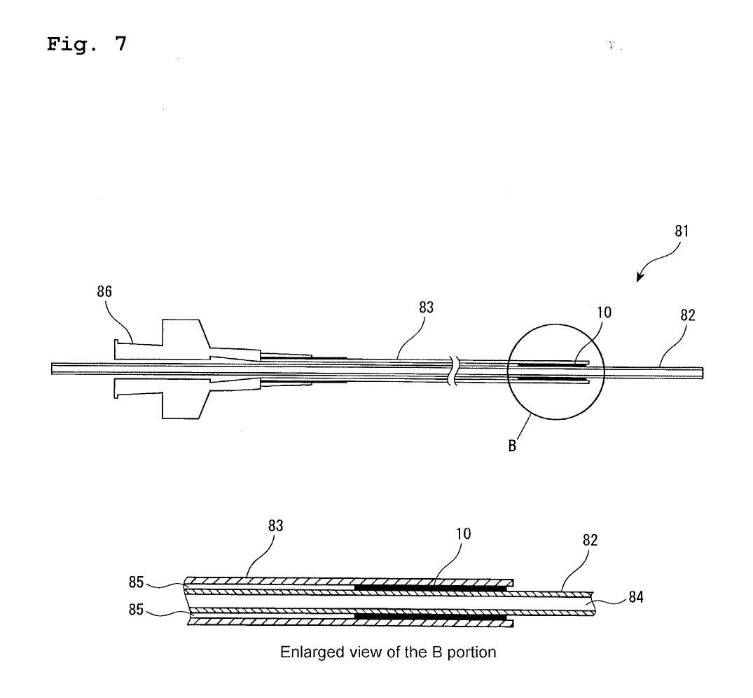

FIG. 7 shows an example of the stent introducer 81. As shown in FIG. 7, the stent introducer 81 is comprised of an inner cylinder portion 82 provided inside, and an outer cylinder portion 83 provided outside the inner cylinder portion 82, the guide wire 51 is passed through a through hole 84 inside the inner cylinder portion 82, and the stent 10 is loaded into a cavity 85 between the inner cylinder portion 82 and the outer cylinder portion 83. In addition, an operating portion 86 is provided at the base end of the stent introducer 81. Since the outside diameter of the outer cylinder portion 83 of the stent introducer 81 is smaller than the inside diameter of the carrier catheter 71, it is possible to introduce the stent introducer 81 into the carrier catheter 71.

(3) Insertion of a Pusher and Pushing of the Stent

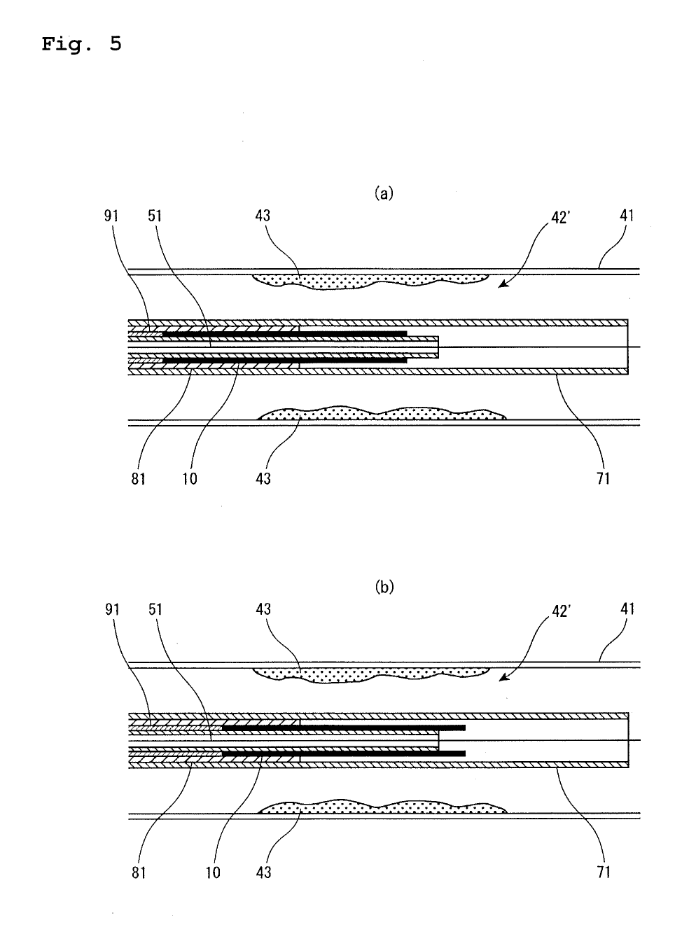

Next, as shown in FIG. 5(a), a pusher 91 is pierced with the guide wire 51 to move forward, and inserted into the stent introducer 81.

The pusher 91 is comprised of a catheter provided at its base end with an operating portion, and the inside diameter of the pusher 91 is larger than the outside diameter of the inner cylinder portion 82 of the stent introducer 81, while the outside shape of the pusher 91 is smaller than the inside diameter of the outer cylinder portion 83 of the stent introducer 81. Accordingly, it is possible to insert in the cavity 85 of the stent introducer 81. When the pusher 91 is moved forward further from the state of FIG. 5(a), as shown in FIG. 5(b), the pusher 91 pushes the stent 10 out of the cavity 85 of the stent introducer 81.

(4) Release and Placement of the Stent

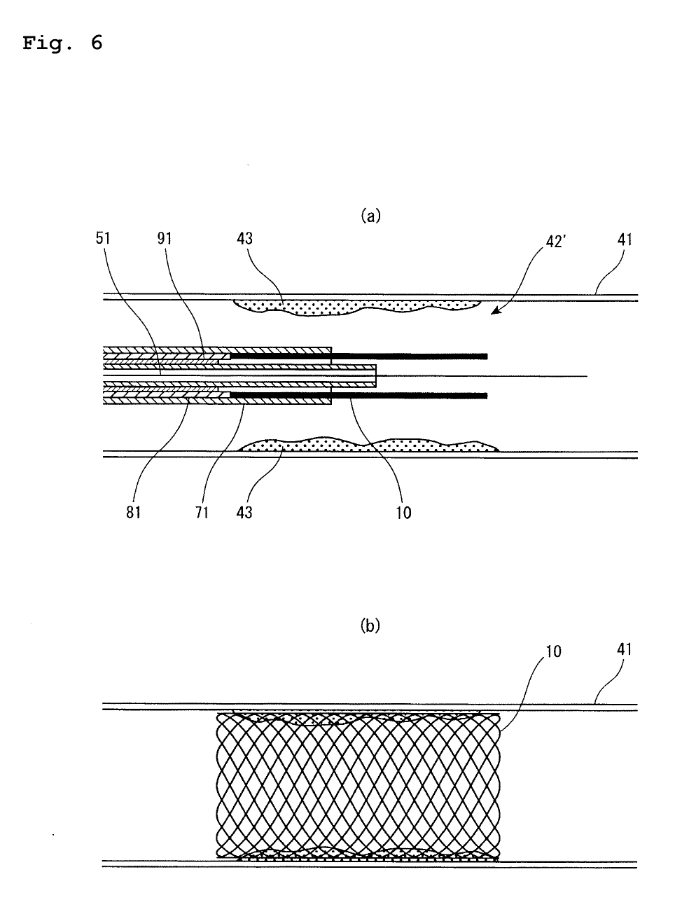

Next, as shown in FIG. 6(a), after the stent 10 is pushed out of the stent introducer 81, the carrier catheter 71 is backed.

Then, as shown in FIG. 6(b), the stent 10 is released from the carrier catheter 71, and expands inside the blood vessel 41. Subsequently, the carrier catheter 71, stent introducer 81, pusher 91 and guide wire 51 are removed from the body. By the above-mentioned means, it is possible to secure the blood stream of the stenosed site 42.

In addition, up to here, as the shape of a stent, the straight cylindrical stent 10 is described as an example, and the invention is applicable to stents in various shapes according to the purpose, target site, etc. Examples of other shapes will be shown below.

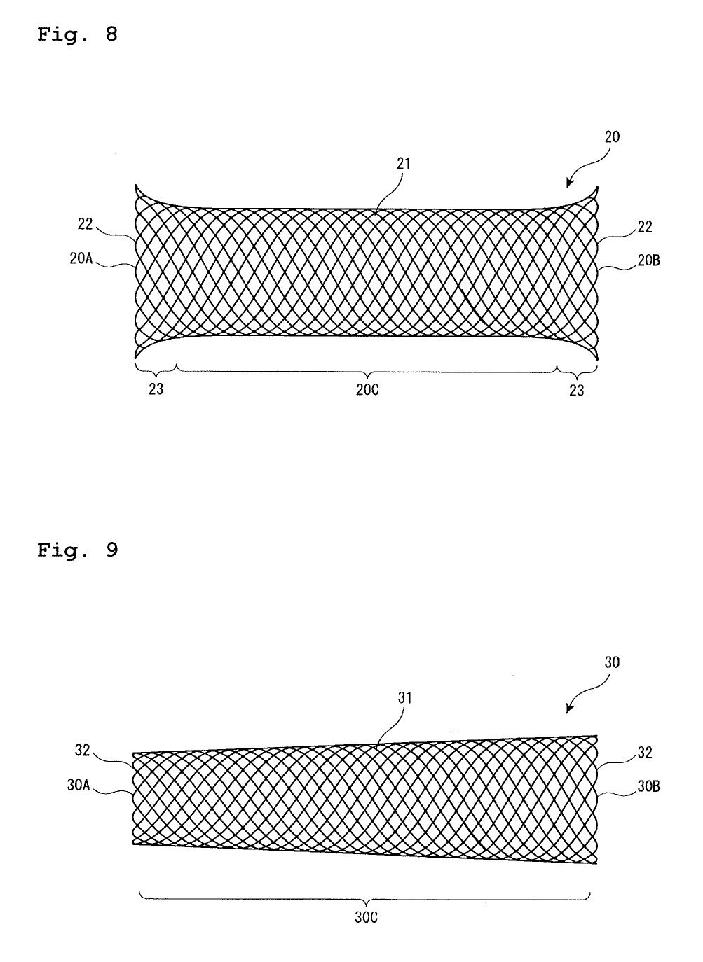

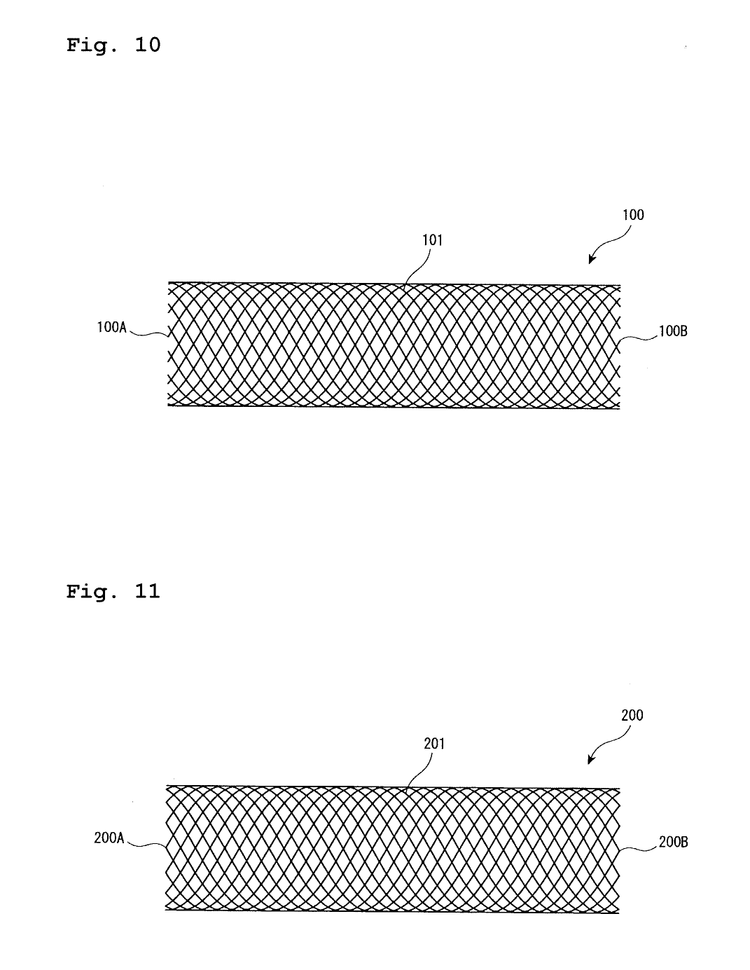

FIG. 8 is a schematic view showing a stent in the opposite-end broad shape. As shown in FIG. 8, in this stent 20, the diameters of opposite end portions 20A, 20B are larger than the diameter of the side portion 20C, and the stent thus has certain diameters in the center and its vicinities of the side portion 20C, and is broadened gradually toward the opposite end portions 20A, 20B in the shape of a flare in vicinities of the opposite end portions 20A, 20B (flare portions 23).

FIG. 9 is a schematic view showing a stent in the tapered shape. As shown in FIG. 9, this stent 30 is formed in the tapered shape in the side portion 30C so that the diameter increases gradually toward the other end portion 30B from one end portion 30A.

In these stents 20, 30, as in the stent 10 shown in FIG. 1, the wires 21, 31 are positioned in the side portions 20C, 30C without being positioned in the end portions 20A, 20B, 30A, 30B, and in the opposite end portions 20A, 20B, 30A, 30B, the wires 21, 31 form curved portions 22, 32 bent in the shape of a curve with roundness so as not to be folded and protrude.

Thus, the invention is applied to stents in various shapes, and is capable of being modified according to various purposes and target sites.

In addition, in the aforementioned description, the stent formed of only the wire is described as an example, but when necessary, it is possible to apply a resin coating, drug coating (DES; Drug-Eluting Stent), etc. The DES is obtained by coating the stent with a drug for inhibiting cell proliferation or the like, the drug elutes slowly in the body, and it is thereby possible to prevent intimae from being excessively formed, and to inhibit re-stenosis effectively.

Further, in the above-mentioned description, the cerebral blood vessel is described as an example, but the stent placement is not limited to the cerebral blood vessel, and is applicable to general lumens (for example, trachea, esophagus, duodenum, large intestine, biliary tract, etc.) of the human body, as well as blood vessels of other sites. Therefore, the stent of the invention is also applicable to general lumens of the human body in the size corresponding to the site to treat. For example, in the case of placing in the cerebral blood vessel to use, it is the most suitable that the outside diameter of the stent 10 is about 5 mm.

By configuring as described above, according to the stent of the invention, it is possible to prevent the wall of a lumen from being injured and to reduce the risk of stenosis of the lumen.

The Embodiments of the invention are described as mentioned above, but the invention is not limited to the above-mentioned Embodiments, is capable of being modified in various manners based on the subject matter of the invention, and is not intended to exclude the modifications from the scope of the invention.

### INDUSTRIAL APPLICABILITY

The invention relates to a stent that is placed in a lumen of the human body to prevent the occurrence of occlusion inside the lumen and breakage of the lumen, and has industrial applicability.

### REFERENCE SIGNS LIST

10 Stent

10A End portion

10B End portion

10C Side portion

11 Wire

12 Curved portion

20 Stent

20A End portion

20B End portion

20C Side portion

21 Wire

22 Curved portion

23 Flare portion

30 Stent

30A End portion

30B End portion

30C Side portion

31 Wire

32 Curved portion

1. A stent in the shape of a mesh that is placed and used inside a lumen such as a blood vessel of the human body,
wherein the mesh is formed of a single or plurality of pieces of wire material having elasticity, and the wire material bends in the shape of a curve without being folded in end portions of the stent, and is formed in the shape of a cylinder.

2. The stent according to claim 1, wherein the end of the wire material is formed to be positioned in a side portion except end portions of the shape of a cylinder.

3. The stent according to claim 1, wherein the stent is formed in the shape of a cylinder in which diameters are almost the same over from one end portion to the other end portion.

4. The stent according to claim 1, wherein the stent is formed in an opposite-end broad shape in which diameters in opposite end portions are larger than a diameter of a side portion close to the center.

5. The stent according to claim 1, wherein the stent is formed in a tapered shape in which a diameter in one end portion is larger than a diameter in the other end portion.

6. The stent according to claim 1, wherein the wire material is a wire made of gold, platinum, or tungsten, or a wire comprised of nickel titanium coated with gold or platinum.

7. The stent according to claim 5, wherein an outside diameter of the shape of a cylinder is formed into 5 mm, and the stent is placed and used inside a cerebral blood vessel.
